(19) United States
(12) Patent Application Publication
(10) Pub. No.: US 2026/0289775 A1
(43) Pub. Date: Sep. 24, 2026

(54) DETECTION OF DIFFUSE RETINAL THICKENING (DRT) USING OPTICAL COHERENCE TOMOGRAPHY (OCT) IMAGES

(71) Applicants: Genentech, Inc., South San Francisco, CA (US); Hoffmann-La Roche Inc., Little Falls, NJ (US)

(72) Inventors: Dimitrios DAMOPOULOS, Bern (CH); Thomas Felix ALBRECHT, Basel (CH); Daniela Ferrara CAVALCANTI, Marlborough, MA (US); Huanxiang LU, Préverenges (CH); Michael H. CHEN, Oakland, CA (US)

(21) Appl. No.: 19/686,159

(22) Filed: May 22, 2026

Related U.S. Application Data
(63) Continuation of application No. PCT/US24/59175, filed on Dec. 9, 2024.
(60) Provisional application No. 63/607,562, filed on Dec. 7, 2023.
(60) Provisional application No. 63/641,777, filed on May 2, 2024.

(51) Int. Cl. G06T 7/00; G06N 20/00; G06T 7/11; G06T 12/10

(52) U.S. Cl. CPC G06T 7/0012; G06N 20/00; G06T 7/11; G06T 12/10; G06T 2207/10101; G06T 2207/20081; G06T 2207/20084; G06T 2207/30041

(57) ABSTRACT

A method and system for detecting the presence of diffuse retinal thickness (DRT) in optical coherence tomography (OCT) images. Detecting the presence of DRT in OCT images includes receiving an OCT imaging data for a retina of a subject and forming an image input for a machine learning model (e.g., a deep learning model) using the OCT imaging data. A machine learning model is used to generate a diffuse retinal thickness (DRT) detection output based on the image input. The DRT detection output indicates whether a presence of DRT is detected in the retina of the subject.

### CROSS-REFERENCE TO RELATED APPLICATIONS

This application is a continuation of International Application No. PCT/US2024/059175, filed Dec. 9, 2024, which is related to and claims the benefit of the priority date of U.S. Provisional Application 63/607,562, filed Dec. 7, 2023, entitled “Detection of Diffuse Retinal Thickening (DRT) Using Optical Coherence Tomography (OCT) Images” and U.S. Provisional Application 63/641,777, filed May 2, 2024, entitled “Detection of Diffuse Retinal Thickening (DRT) Using Optical Coherence Tomography (OCT) Images,” each of which is incorporated herein by reference in its entirety.

### FIELD

This application relates to the detection of diffuse retinal thickening (DRT), and more particularly, to the automated classification of optical coherence tomography (OCT) imaging data as evidencing DRT or not evidencing DRT.

### BACKGROUND

Retinal diseases, such as diabetic macular edema (DME) and age-related macular degeneration (AMD), are leading causes of vision loss in subjects 50 years and older. Some subjects with DME or AMD can develop diffuse retinal thickening (DRT), in which there is swelling of the retina with areas of lower reflectivity, due to fluid accumulation within the retina. Upon entering the retina, the fluid may distort the vision of a subject immediately. Over time, the fluid can damage the retina itself, for example, by causing the loss of photoreceptors in the retina.

### SUMMARY

In one or more embodiments, a method for detecting the presence of diffuse retinal thickening (DRT) in optical coherence tomography (OCT) imaging data is provided. OCT imaging data may be received for a retina of a subject. A first image input may be formed for a machine learning model (e.g., a deep learning model) using the OCT imaging data. The machine learning model may be used to generate a diffuse retinal thickness (DRT) detection output based on the first image input. The DRT detection output indicates whether or not a presence of DRT is detected in the retina of the subject.

In one or more embodiments, a method of approximating an area of DRT present in an OCT imaging data is provided. OCT imaging data may be received for a retina of a subject. An image input may be formed for a machine learning model (e.g., a deep learning model) using the OCT imaging data. The machine learning model may be used to generate a diffuse retinal thickness (DRT) detection output based on the first image input. An area of DRT present in the image input may be approximated.

In one or more embodiments, a method of approximating a volume of DRT present in an OCT imaging data is provided. OCT imaging data may be received for a retina of a subject. An image input may be formed for a machine learning model (e.g., a deep learning model) using the OCT imaging data. The machine learning model may be used to generate a diffuse retinal thickness (DRT) detection output based on the first image input. A volume of DRT present in the image input may be approximated.

In one or more embodiments, a system comprises at least one data processor; and at least one memory storing instructions, which when executed by the at least one data processor, result in operations comprising any one or more of the methods described herein or a portion thereof.

In one or more embodiments, a non-transitory computer readable medium storing instructions is provided, which when executed by at least one data processor, result in comprising any one or more of the methods described herein or a portion thereof.

### BRIEF DESCRIPTION OF DRAWINGS

For a more complete understanding of the principles disclosed herein, and the advantages thereof, reference is now made to the following descriptions taken in conjunction with the accompanying drawings, in which:

FIG. 1 is a block diagram of a diffuse retinal thickness (DRT) detection system, in accordance with various embodiments.

FIG. 2 is a block diagram of a DRT approximation model for approximating DRT area, in accordance with various embodiments.

FIG. 3 is a block diagram of a DRT approximation model for approximating DRT volume, in accordance with various embodiments.

FIG. 4 is a flowchart for detecting DRT presence, in accordance with various embodiments.

FIG. 5 is a flowchart for approximating an area of DRT, in accordance with various embodiments.

FIG. 6 illustrates example images for approximating an area of DRT, in accordance with various embodiments.

FIG. 7 is a flowchart for approximating a volume of DRT, in accordance with various embodiments.

FIGS. 8A and 8B illustrate example images for approximating a volume of DRT, in accordance with various embodiments.

FIG. 9 is a block diagram of a computer system, in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

### I. Overview

The embodiments described herein recognize that detecting presence of diffuse retinal thickening (DRT) may be important for managing retinal diseases such as, for example, diabetic macular edema (DME) and age-related macular degeneration (AMD). For example, being able to accurately and reliably detect the presence of DRT may be helpful in managing the treatment of DME or AMD. For example, having an automated system and method for detecting DRT presence may allow generation of a personalized treatment regimen for a subject with retinal disease, for mitigating retinal damage, and for understanding a subject's retinal disease pathogenesis. Optical coherence tomography (OCT) imaging may be used to detect DRT in retinas affected by retinal diseases such as age-related macular degeneration (AMD) and diabetic macular edema (DME). OCT is an imaging technique in which light is directed at a biological sample (e.g., biological tissue) and the light that is reflected from features of that biological sample is collected to capture two-dimensional or three-dimensional, high-resolution cross-sectional images of the biological sample.

DRT is a type of edema that, contrary to commonly measured retinal fluids, is diffuse in nature and as such, difficult for experts (e.g., human graders) to identify or delineate. In some cases, DRT may be indicated by diffuse retinal fluid (e.g., intraretinal fluid, subretinal fluid, subretinal pigment epithelial fluid, etc.) that causes an increased retinal thickness (>200 microns height and >200 microns width) with areas of hyporeflectivity relative to other parts of the retina. While OCT images enable visualizing such diffuse retinal fluid, delineating the presence of DRT may be difficult for human graders, because in contrast to intraretinal fluid cysts, there are no well-defined cyst walls visible on an OCT image. As such, manual analysis of OCT images by human graders may lack consistency both intra- and inter-graders. Accordingly, manual analysis of OCT images by human graders may be time-consuming and prone to error. Additionally, for these same reasons, segmentation of DRT in OCT images by human graders is even more difficult than classification of DRT by human graders.

Thus, the embodiments described herein recognize that it may be desirable to have systems and methods for automating the detection of DRT. For example, it may be desirable to have systems and methods of accurately and reliably classifying OCT images as evidencing DRT (e.g., being DRT positive) or not evidencing DRT (e.g., being DRT negative). Accordingly, the embodiments described herein provide one or more technical benefits, which may include, for example, without limitation, improving the performance (e.g., accuracy) of a model and/or improving the performance (e.g., accuracy) of a computer system that is specially configured to run the model to perform automated classification of DRT (e.g., the absence or presence of DRT) on OCT images.

Recognizing and taking into account the importance and utility of a methodology and system that can provide the improvements described above, the specification describes various embodiments for automated DRT detection using OCT imaging data. More particularly, the specification describes various embodiments of methods and systems for accurately and reliably classifying OCT imaging data, using a machine learning system (e.g., a deep learning system, which may be a neural network system), as evidencing or not evidencing the presence of DRT in a retina.

### II. Example DRT Detection System

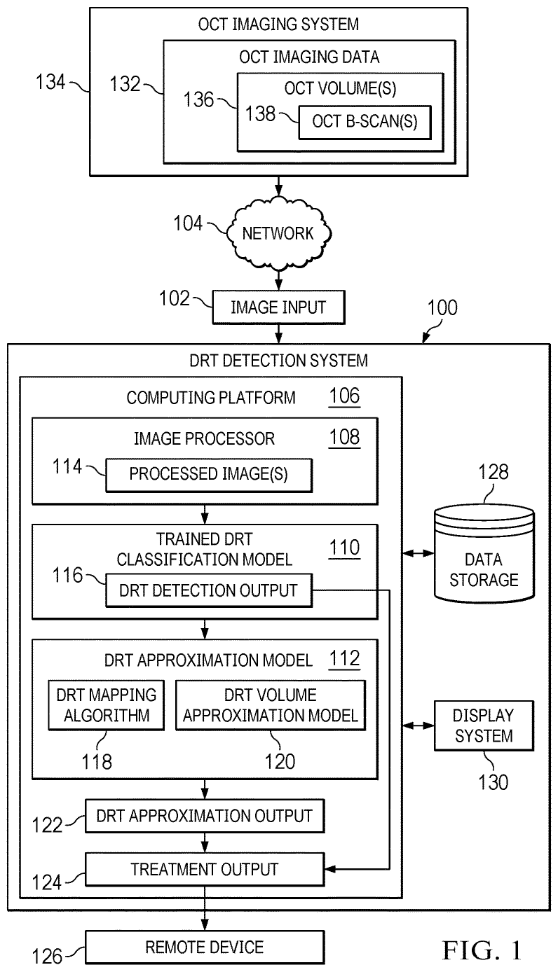

FIG. 1 is a block diagram of a DRT detection system 100 in accordance with various embodiments. The DRT detection system 100 is used to detect the presence of DRT in the retinas of subjects using image input 102, which may be received or accessed via a network 104. In some embodiments, the retina is a healthy retina. In other embodiments, the retina is one that has been diagnosed with or is suspected of having a retinal disease. For example, the diagnosis may be one of age-related macular degeneration (AMD), diabetic macular edema (DME), or some other type of retinal disease. In some embodiments, the DRT detection system 100 detects the presence of DRT in a patient, provides an approximation of DRT area in a patient, and/or provides an approximation of DRT volume in a patient.

As illustrated in FIG. 1, the DRT detection system 100 includes a computing platform 106 configured to store and execute an image processor 108, a trained DRT classification model 110, and a DRT approximation model 112. While the image processor 108, the trained DRT classification model 110, and the DRT approximation model 112 are illustrated as being stored and executed using the same computing platform (i.e., the computing platform 106), in some embodiments, one or more of the image processor 108, the model 110, and the DRT approximation model 112 are stored and executed using a computing platform that is different from the computing platform 106. Generally, the image processor 108 receives or accesses the image input 102 and generates processed image(s) 114. The preprocessed image(s) 114 are inputs to the trained DRT classification model 110, which uses the processed image(s) 114 to generate a DRT detection output 116. As illustrated, the DRT approximation model 112 may include a DRT mapping algorithm 118 or a DRT volume approximation model 120. In some examples, the DRT approximation model 112 generates an DRT approximation output 122, which may be used to generate a treatment output 124, which is sent to a remote device 126 via the network 104. In some examples, however, the treatment output 124 is based on the DRT detection output 116 without reference to the DRT approximation output 122.

The DRT detection system 100 also includes a data storage 128 and a display system 130. The data storage 128 and display system 130 are each in communication with the computing platform 106. In some examples, the data storage 128, display system 130, or both may be considered part of or otherwise integrated with the computing platform 106. Thus, in some examples, the computing platform 106, the data storage 128, and the display system 130 may be separate components in communication with each other, but in other examples, some combination of these components may be integrated together.

As illustrated, the image input 102 may include OCT imaging data 132, which may be generated using an OCT imaging system 134 or OCT scanner. The OCT imaging system 134 can be a large tabletop configuration used in clinical settings, a portable or handheld dedicated system, or a “smart” OCT system incorporated into user personal devices such as smartphones. In some cases, the OCT imaging system 134 may include an image denoiser that is configured to remove noise and other artifacts from a raw OCT volume image to generate an OCT volume. In one or more embodiments, the OCT imaging data 132 includes OCT volume(s) 136 for a retina of a subject. Each of the OCT volume(s) 136 may be comprised of a plurality of OCT B-scans 138 of the retina of the subject. The plurality of OCT B-scans 138 may include, for example, without limitation, 10s, 100s, 1000s, 10,000s, or some other number of OCT B-scans. An OCT B-scan may also be referred to as an OCT slice image or a cross-sectional OCT image.

Although only one of each of OCT imaging system 134 and the DRT detection system 100 is shown, there can be more than one of each in other embodiments. Further, although FIG. 1 shows the OCT imaging system 134 and the DRT detection system 100 as two separate components, in some embodiments, the OCT imaging system 134 and the DRT detection system 100 may be parts of the same system (e.g., and maintained by the same entity such as a health care provider or clinical trial administrator). In some cases, a portion of the DRT detection system 100 may be implemented as part of OCT imaging system 134. For example, the DRT detection system 100 may be configured to run as a module implemented using a processor, microprocessor, or some other hardware component of OCT imaging system 134. In still other embodiments, the DRT detection system 100 may be implemented within a cloud computing system that can be accessed by or otherwise communicate with the OCT imaging system 134.

In one embodiment, the image processor 108 is configured or programmed to receive and perform a set of processing operations on the OCT imaging data 132, which is the image input 102, to form the processed images 114. The OCT imaging data 132 may be sent as input into the image processor 108, retrieved by the image processor 108 from storage, or accessed in some other manner. The set of processing operations may include, for example, without limitation, at least one of a normalization operation, a scaling operation, a resizing operation, a horizontal flipping operation, a vertical flipping operation, a cropping operation, a rotation operation, a noise filtering operation, or some other type of preprocessing operation. The image processor 108 may be implemented using hardware, software, firmware, or a combination thereof. In one or more embodiments, the image processor 108 may be implemented within the computing platform 106 but in other embodiments at least a portion of (e.g., a module of) the image processor 108 is implemented within the OCT imaging system 134.

In some embodiments, the trained DRT classification model 110 is a machine learning or a deep learning model that is trained to classify image input, such as one or more of the processed image(s) 114, based on whether the presence of DRT is detected in the retina of the subject. For example, the trained DRT classification model 110 may output the DRT detection output 116, which may include a classification of one or more of the processed image(s) 114 as being DRT positive (e.g., evidencing the presence of DRT) or DRT negative (e.g., not evidencing the presence of DRT). The DRT detection output 116 may be a probability value indicating the probability that DRT is present in the retina. The probability value may be quantitative (e.g., percentages) or qualitative (e.g., DRT positively present, DRT possibly present, DRT positively absent). In some examples, DRT detection output 116 is a binary output that signals that DRT is present in the retina or that DRT is absent in the retina. The deep learning model may be implemented using one or more neural network systems. For example, the deep learning model may be implemented using any number of or combination of neural networks. In one or more embodiments, the deep learning model includes a convolutional neural network (CNN), which itself may include one or more neural networks.

In some embodiments, the trained DRT classification model 110 was trained using training data that included a plurality of OCT B-scan images of DME and AMD patients, which had been annotated by human graders to classify DRT within the OCT image. In an example training, the training data included 5,133 B-scans of 276 patients that were annotated by trained graders to classify the OCT images into one of four categories of DRT: positively present; possibly present; positively absent; ungradable (due to poor image quality). In this example, 90% of the images were graded by four graders and 98% of the images were graded by more than two graders. In this example training, there were 19,993 annotations, which were grouped together. For example, and due to the rarity of the possibly present and ungradable gradings, OCT images with DRT classified as positively present or possibly present were grouped together and classified as DRT-positive. Similarly, OCT images with DRT classified as positively absent or ungradable were grouped together and classified as DRT-negative. In this example training and for the validation set, the category chosen by the graders' majority was treated as the ground truth resulting in 490 images graded as DRT-negative and 293 as DRT-positive. The dataset include a single image graded by majority as ungradable was omitted.

In some embodiments, the trained DRT classification model 110 was trained using a splitting strategy. In the example training, the hyperparameters of an example convolutional neural network (CNN) for this binary classification task (e.g., InceptionV3, ImageNet initialization) were selected with a cross-validation nested in the training set. In this example training, a five-fold cross-validation was used. The training and interference on the gradable test was repeated ten times to estimate the variance. Over 10 training repetitions, the example CNNs classified the images of the validation set with an average area under the receiver operator characteristic (AUROC) of 99.2% (0.4% SD). In the DME validation subset (424 of the 783 images) the AUROC was 98.5% (0.6% SD).

The treatment output 124 may include identifying a patient as a patient that is at high risk of experiencing DRT or a patient that is experiencing DRT. In some embodiments, such identification is based on the DRT detection output 116 and/or the DRT approximation output 122. In some embodiments, the treatment output 124 also includes administering or recommending the administration, based on the identification of the patient as a patient that is at high risk of experiencing DRT or a patient that is experiencing DRT, of an appropriate treatment. In some embodiments, the appropriate treatment may include an anti-VEGF therapy, such as ranibizumab, aflibercept, or bevacizumab.

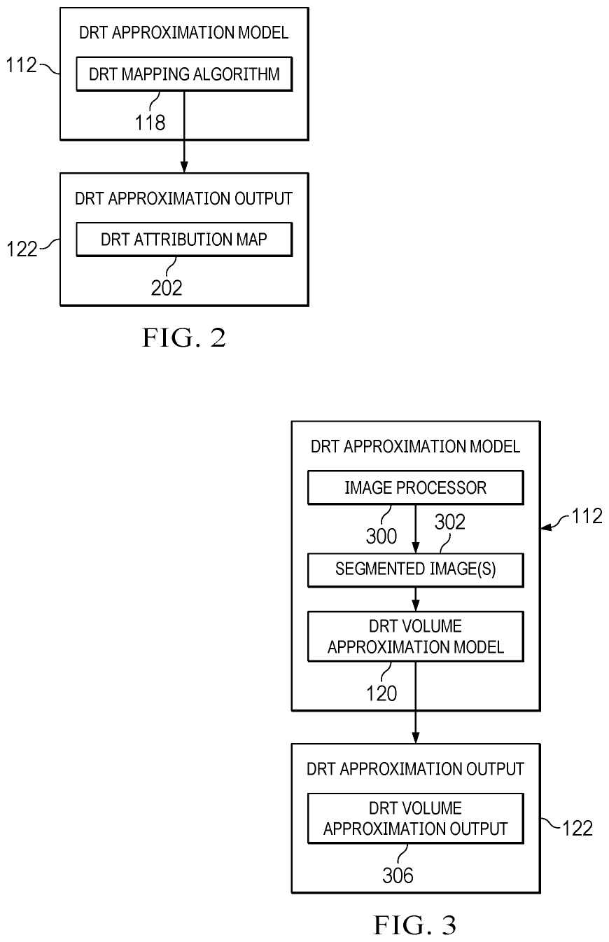

FIG. 2 is a block diagram of the DRT approximation model 112 and the DRT approximation output 122 in accordance with various embodiments. The DRT approximation model 112 is used to generate DRT approximation output 122 for retinas of subjects classified as being DRT-positive. In some embodiments, the DRT approximation model 112 may comprise a DRT mapping algorithm 118. The DRT may include, but is not limited to, gradient-weighted Class Activation Mapping (Grad-CAM), a technique that provides “visual explanations” in the form of heatmaps for the decisions that a deep learning model makes when performing predictions. That is, Grad-CAM may be implemented for a trained deep learning model to generate attribution maps or heatmaps of OCT B-scans in which the heatmaps indicate (e.g., using colors, outlines, annotations, etc.) the regions or locations of the OCT B-scans that the neural network model uses in making classifications of DRT for the retinas shown in the OCT B-scans. In one or more embodiments, Grad-CAM may determine the degree of importance of each pixel in an OCT B-scan to the DRT classification output generated by the trained DRT classification model 110. Additional details about Grad-CAM may be found in R. R. Selvaraju et al., “Grad-CAM: Visual Explanations from Deep Networks via Gradient-based Localization,” Arxiv: 1610.02391 (2017), which is incorporated by reference herein in its entirety. Other non-limiting examples of attribution mapping techniques include class activation mappings (CAMs), SmoothGrad, the Low-Variance Gradient Estimator for Variational Inference (VarGrad), and/or the like, or a combination thereof.

DRT mapping algorithm 118 may generate, as DRT approximation output 122, a DRT attribution map 202. DRT attribution map 202 indicates (e.g., via a heatmap) the degree of importance for the various pixels (or regions) of the image input 102 with respect to DRT detection output 116. In other words, DRT attribution map 202 indicates the level of contribution of the various pixels of the image input 102 to the DRT detection output 116 generated by the trained DRT classification model 110. The DRT attribution map 202 may visually indicate (e.g., via color, highlighting, shading, pattern, outlining, text, annotations, etc.) the regions of the corresponding OCT B-scan of the image input 102 that were most impactful to the trained DRT classification model 110 for determining the DRT detection output 116.

In one or more embodiments, the DRT attribution map 202 may be used to quantify the number of high-importance pixels in the image input 102 to provide an approximate area of DRT. In various embodiments, the DRT attribution map 202 may be used to locate centers of the connected components of high-importance pixels to provide approximated DRT location(s) in the OCT B-scan of the image input 102.

In some embodiments, the treatment output 124 may be generated using the DRT attribution map 202. For example, the treatment output 124 may be generated based on the approximate area of DRT as quantified by counting the number of high-importance pixels in the image input 102. In other examples, the treatment output 124 may be generated based on changes in the approximate area of DRT in image inputs captured at timepoints after a baseline timepoint, relative to the baseline timepoint, of a retina of a patient. In such examples, the treatment output 124 may further be generated based on changes in the area of DRT with respect to the approximated DRT locations captured at timepoints after the baseline timepoint, relative to the baseline timepoint, of the retina of the patient.

FIG. 3 is a block diagram of the DRT approximation model 112 and the DRT approximation output 122 in accordance with various embodiments. The DRT approximation model 112 is used to generate DRT approximation output 122 for retinas of subjects classified as being DRT-positive. In some embodiments, the DRT approximation model 112 includes an image processor 300 that is an OCT segmentation system that generates segmented image(s) 302 using the processed image(s) 114 of FIG. 1. However, the image processor 300 may be a separate component from the DRT approximation model 112 and be in communication with the DRT approximation model 112. The segmented image(s) 302 are then used to identify various retinal elements within the OCT B-scan of the image input 102 with respect to DRT detection output 116. For example, one or more of the segmented image(s) 302 may be generated from OCT imaging data according to one or more techniques as described in International Publication No. WO2023205511A1, which is incorporated by reference herein in its entirety. Moreover, and in some embodiments, the image processor 300 is or includes one or more of the systems for automated retinal segmentation as described in International Publication No. WO2023205511A1. A retinal element may be comprised of at least one of a retinal layer element or a retinal pathological element. Detection and identification of one or more retinal layer elements may be referred to as layer element (or retinal layer element) segmentation. Detection and identification of one or more retinal pathological elements may be referred to as pathological element (or retinal pathological element) segmentation. The image processor 300 identifies one or more retinal elements on the segmented image(s) 302 using one or more graphical indicators. For example, one or more color indicators, shape indicators, pattern indicators, shading indicators, lines, curves, markers, labels, tags, text features, other types of graphical indicators, or a combination thereof may be used to identify the portion(s) (e.g., by pixel) of an OCT image that have been identified as a retinal element. In some embodiments, the volume of the segmented retinal elements may be used to approximate the volume of the identified DRT. For example, the volume of the retinal pathological elements (e.g., any cystic intraretinal fluid (IRF) and/or subretinal fluid (SRF)) may be subtracted from the volume between the two retinal layer elements (e.g., retinal layers, such as for example without limitation, the internal limiting membrane (ILM) and the Bruch's Membrane (BM)). The resulting volume approximation may be used as an estimate for the DRT volume as well as any volume associated with healthy tissue. In some embodiments, the DRT volume approximation model 120 calculates the DRT volume approximation output 306. The DRT volume approximation model 120 may be implemented using hardware, software, firmware, or a combination thereof. The DRT volume approximation output 306 may be the DRT approximation output 122. In some embodiments, this combined volume of DRT and healthy tissue may be assessed over a baseline timepoint and one or more timepoints after the baseline timepoint, in order to assess changes to DRT volume over time. In other examples, the treatment output 124 may be generated based on changes in the DRT volume approximation output 306 in image inputs captured at timepoints after a baseline timepoint, relative to the baseline timepoint, of a retina of a patient.

In some embodiments, repeatability of the quantitative measures (e.g., volume measurements, layer thickness, etc.) derived from the segmented images generated according to one or more automated segmentation techniques as described in International Publication No. WO2023205511A1 may be assessed. For example, the repeatability standard deviation (SD) and/or coefficient of variation (CV) for thickness measures may be assessed to confirm whether the thickness measures are sufficiently repeatable for use in the DRT volume approximations as described above. As another example, the repeatability standard deviation (SD) for fluid volume measures may be assessed to confirm whether it is within the limit of detection for healthcare providers.

In various embodiments, repeatability of volume measurements derived from the segmented images generated according to one or more automated segmentation techniques as described in International Publication No. WO2023205511A1 may be assessed to ensure the quantitative measures (e.g., volume measurements), used in DRT volume approximations as described above, are accurate and consistent. In some embodiments, repeatability of the quantitative measures may be assessed using repeated OCT B-scans.

In an example assessment of repeatability of quantitative measures, OCT B-scans from a clinical trial including repeated OCT scans were used. In this example, two comparable OCT scans per eye were acquired for almost every patient visit (one macular cube with 97 OCT B-scans and one macular cube with 49 OCT B-scans), such that 10,021 image pairs were obtained for 225 unique eyes. In this example, to approximate taking the same scan twice, the macular cubes with 97 OCT B-scans were subsampled down to 49° C. T B-scans by including only odd number OCT B-scans, to simulate repeated scans of the same density. Automated segmentation techniques as described in International Publication No. WO2023205511A1 were then performed to segment retinal elements (i.e., retinal layer elements and retinal pathological elements) and extract quantitative measures. In this example, thickness of the central subfield layer (also referred to as central subfield thickness, or CST) and volume of intraretinal fluid (IRF) and subretinal fluid (SRF) elements were used as quantitative measures. One image pair per eye was randomly selected to estimate repeatability using independent observations and to compute the repeatability standard deviation (SD) and repeatability coefficient of variation (CV). The results of this example repeatability assessment are provided in Table 1 below.

TABLE 1
Example Repeatability Assessment of CST and Fluid
(IRF and SRF) Volume Measurements

| | | Repeatability | Repeatability |
|---|---|---|---|
| Measurement | Region | SD | CV % |
| CST (ILM to BM) | Central 1 mm | 5.89 μm | 1.64 |
| CST (ILM to BM) | Central 3 mm | 1.87 μm | 0.49 |
| CST (ILM to IB_RPE) | Central 1 mm | 4.58 μm | 1.81 |
| CST (ILM to IB_RPE) | Central 3 mm | 0.62 μm | 0.27 |
| IRF volume | Central 1 mm | 0.44 nl | N/A |
| IRF volume | Central 3 mm | 0.95 nl | N/A |
| SRF volume | Central 1 mm | 0.34 nl | N/A |
| SRF volume | Central 3 mm | 0.82 nl | N/A |

As seen in Table 1, the results of the example repeatability assessment indicate the repeatability SD for fluid volumes is low, and indicate the repeatability CVs for fluid volumes are N/A because many scans with no fluid volume cause a zero denominator in the CV formula. In this example, the repeatability SD and CV for thickness measures are comparable to repeatability SD and CV for thickness measures as measured by other devices used to measure layer thickness measures, and repeatability SD for fluid volume is likely within the limit of detection for healthcare providers.

In some embodiments, the DRT detection system detects DRT in patients with better accuracy and consistency than expert human graders. The DRT detection system 100 provides a technical effect of improving accuracy, reducing the overall computing resources, and/or reducing the time needed to detect DRT in subjects. Further, using the DRT detection system 100 may allow treatment outcomes in subjects to be generated more efficiently and accurately as compared to other methods and systems.

In some embodiments, the DRT detection system 100 provides a technical improvement to the field of DRT detection and/or the technical field of generating DRT treatment outputs. As noted above, the DRT detection system 100 detects DRT in patients with better accuracy and consistency than expert human graders and may reduce the overall computing resources and/or time needed to detect DRT in subjects.

### III. Example Process for Classification of DRT Presence

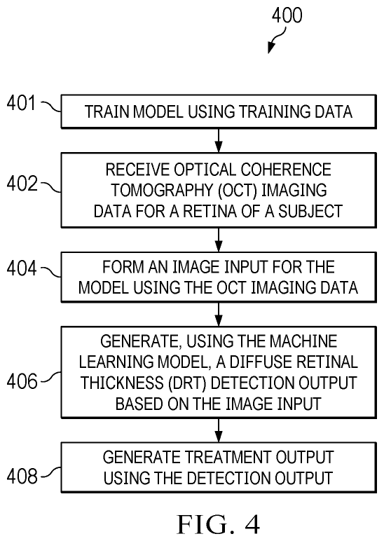

FIG. 4 is a flowchart of a process 400 for detecting diffuse retinal thickness (DRT) in OCT imaging in accordance with various embodiments. DRT may be a biomarker associated with retinal disease such as, for example, DME or AMD. In various embodiments, the process 400 is implemented using the DRT detection system 100 described in FIG. 1.

Process 400 may optionally include the step 401 of training a model (e.g., a deep learning model). Training the model may include the example training of the CNN model that resulted in the trained DRT classification model 110 of FIG. 1. The model may include a neural network system such as, for example, a convolutional neural network (CNN). The model may be trained to process OCT images and classify the OCT images as evidencing a presence of DRT or not evidencing a presence of DRT. For example, the model may classify each OCT image as being DRT-positive or DRT-negative.

Step 402 of process 400 includes receiving optical coherence tomography (OCT) imaging data for a retina of a subject. The OCT imaging data may be, for example, OCT imaging data 132 in FIG. 1. The retina may be a retina diagnosed with or suspected of having a retinal disease. The retinal disease may be, for example, age-related macular degeneration (AMD), diabetic macular edema (DME), or some other type of retinal disease. In other embodiments, the retina may be a healthy retina or a retina for which no diagnosis has yet been made.

Step 404 of process 400 includes forming an image input for a model using the OCT imaging data. The image input may be, for example, processed image(s) 114 described in FIG. 1. As discussed above, the model may be, for example, model 110 in FIG. 1. Step 404 may be performed in various ways. In one or more embodiments, forming the image input simply includes sending the OCT imaging data as is into the model 110. In other embodiments, forming the image input may include performing a set of preprocessing operations on the OCT imaging data using the image processor 108 of FIG. 1. The set of preprocessing operations may include, for example, without limitation, at least one of a normalization operation, a scaling operation, a resizing operation, a horizontal flipping operation, a vertical flipping operation, a cropping operation, a rotation operation, a noise filtering operation, or some other type of preprocessing operation.

Step 406 of process 400 includes generating, via the model 110, a diffuse retinal thickness (DRT) detection output based on the image input. The DRT detection output may be, for example, the DRT detection output 116 in FIG. 1. In one or more embodiments, DRT detection output may be a probability value indicating the probability that DRT is present in the retina. The probability value may be quantitative (e.g., percentages) or qualitative (e.g., DRT positively present, DRT possibly present, DRT positively absent). In some examples, the DRT detection output may be a binary output that indicates whether the presence of DRT is detected or whether DRT is absent in the retina. In some embodiments, the step 406 also includes, when the DRT detection output indicates detection of DRT, identifying the patient associated with the image input as a patient at high risk of experiencing DRT or as a patient that is experiencing DRT.

Step 408 of the process 400 includes generating a treatment output using the detection output. The treatment output may be, for example, the treatment output 124 of FIG. 1. In one or more embodiments, the treatment output includes administering an appropriate treatment to the patient being identified at high risk of experiencing or as a patient that is experiencing DRT. In some embodiments, appropriate treatment includes an anti-VEGF therapy, such as ranibizumab, aflibercept, or bevacizumab.

In some embodiments, the process 400 detects DRT in patients with better accuracy and consistency than expert human graders. The process 400 provides a technical effect of improving accuracy, reducing the overall computing resources, and/or reducing the time needed to detect DRT in subjects. Further, the process 400 may allow treatment outcomes in subjects to be generated more efficiently and accurately as compared to other methods and systems.

In some embodiments, the process 400 provides a technical improvement to the field of DRT detection and/or the technical field of generating DRT treatment outputs. As noted above, the process 400 detects DRT in patients with better accuracy and consistency than expert human graders and may reduce the overall computing resources and/or time needed to detect DRT in subjects. In some embodiments, the process 400 includes a new combination of steps that results in the technical improvement over conventional DRT detection methods.

### IV. Example Process for Approximation of DRT Area

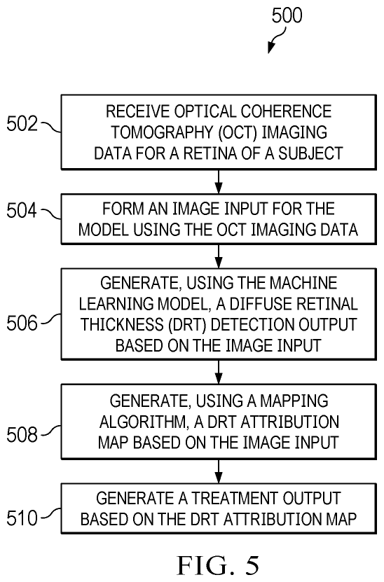

FIG. 5 is a flowchart of a process 500 for approximating an area of DRT in accordance with various embodiments. An area of DRT may be approximated in image inputs which have been classified as DRT-positive, by quantifying the number of high-importance pixels in the image input. In various embodiments, process 500 is implemented using the DRT detection system 100, as described in FIG. 1, and more specifically, using the mapping algorithm 118 as a DRT approximation model 112, as described in FIG. 2.

Step 502 of process 500 includes receiving optical coherence tomography (OCT) imaging data for a retina of a subject. The OCT imaging data may be, for example, OCT imaging data 132 in FIG. 1.

The retina may be a retina diagnosed with or suspected of having a retinal disease. The retinal disease may be, for example, age-related macular degeneration (AMD), diabetic macular edema (DME), or some other type of retinal disease. In other embodiments, the retina may be a healthy retina or a retina for which no diagnosis has yet been made.

Step 504 of process 500 includes forming an image input for a model using the OCT imaging data. The image input may be, for example, image input 102 in FIG. 1. As discussed above, the model may be, for example, trained DRT classification model 110 in FIG. 1. Step 504 may be performed in various ways. In one or more embodiments, forming the image input simply includes sending the OCT imaging data as is into the model. In other embodiments, forming the image input may include performing a set of preprocessing operations on the OCT imaging data. The set of preprocessing operations may include, for example, without limitation, at least one of a normalization operation, a scaling operation, a resizing operation, a horizontal flipping operation, a vertical flipping operation, a cropping operation, a rotation operation, a noise filtering operation, or some other type of preprocessing operation.

Step 506 of process 500 includes generating, via the model, a diffuse retinal thickness (DRT) detection output based on the image input. The DRT detection output may be, for example, DRT detection output 116 in FIG. 1.

The model may include a neural network system such as, for example, a convolutional neural network (CNN). The model may be trained to process OCT images and classify the OCT images as evidencing a presence of DRT or not evidencing a presence of DRT.

In one or more embodiments, DRT detection output may be a probability value indicating the probability that DRT is present in the retina. The probability value may be quantitative (e.g., percentages) or qualitative (e.g., DRT positively present, DRT possibly present, DRT positively absent). In some examples, the DRT detection output may be a binary output that indicates whether the presence of DRT is detected or whether DRT is absent in the retina. For example, the model may classify each OCT image as being DRT-positive or DRT-negative.

Step 508 of process 500 includes generating a DRT attribution map using a DRT mapping algorithm on image input(s) which have been classified as DRT-positive. The DRT mapping algorithm may be, for example, DRT mapping algorithm 118 in FIGS. 1 and 2. The DRT attribution map may be, for example, DRT attribution map 202 in FIG. 2.

The DRT mapping algorithm may include, but is not limited to, gradient-weighted Class Activation Mapping (Grad-CAM), a technique that provides “visual explanations” in the form of heatmaps for the decisions that a deep learning model makes when performing predictions. That is, Grad-CAM may be implemented for a trained deep learning model to generate attribution maps or heatmaps of OCT B-scans in which the heatmaps indicate (e.g., using colors, outlines, annotations, etc.) the regions or locations of the OCT B-scans that the neural network model uses in making classifications of DRT for the retinas shown in the OCT B-scans. In one or more embodiments, Grad-CAM may determine the degree of importance of each pixel in an OCT B-scan to the DRT classification output generated by the trained DRT classification model 110. Additional details about Grad-CAM may be found in R. R. Selvaraju et al., “Grad-CAM: Visual Explanations from Deep Networks via Gradient-based Localization,” Arxiv: 1610.02391 (2017), which is incorporated by reference herein in its entirety. Other non-limiting examples of attribution mapping techniques include class activation mappings (CAMs), SmoothGrad, the Low-Variance Gradient Estimator for Variational Inference (VarGrad), and/or the like, or a combination thereof.

The DRT attribution map indicates (e.g., via a heatmap) the degree of importance for the various pixels (or regions) of the image input with respect to the DRT detection output. In other words, the DRT attribution map indicates the level of contribution of the various pixels of the image input to the DRT detection output generated by trained DRT classification model. The DRT attribution map may visually indicate (e.g., via color, highlighting, shading, pattern, outlining, text, annotations, etc.) the regions of the corresponding OCT B-scan of the image input that were most impactful to the trained DRT classification model for determining the DRT detection output.

In one or more embodiments, the DRT attribution map may be used to quantify the number of high-importance pixels in the image input to provide an approximate area of DRT. In various embodiments, the DRT attribution map may be used to locate centers of the connected components of high-importance pixels to provide approximated DRT location(s) in the OCT B-scan of the image input.

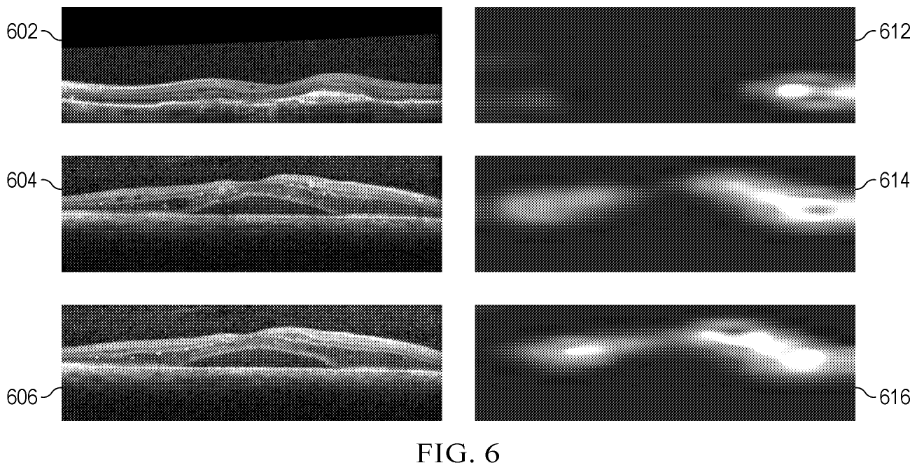

FIG. 6 illustrates an example of three OCT B-scans (602, 604, and 606) which have been processed as image input (e.g., image input 102), and for which the trained DRT classification model (e.g., trained DRT classification model 110) has determined a DRT detection output (e.g., DRT detection output 116) as being DRT positive. FIG. 6 also illustrates an example of three corresponding DRT attribution maps (e.g., DRT attribution map 202), which have been generated by inputting OCT B-scans 602, 604, and 606 into the DRT mapping algorithm. In each of the DRT attribution maps (612, 614, and 616), a dark to light gradient has been used to indicate less importance to most importance, such that light pixels or regions are those that contributed the most (or were most important to) the determination of the DRT detection output as being DRT positive for all three OCT B-scans (602, 604, and 606). In some examples, dark pixels or regions surrounded by light pixels or regions may additionally indicate importance to the determination of the DRT detection output as being DRT positive, as seen on DRT attribution map 614.

Step 510 of process 500 includes generating a treatment output using the DRT attribution map. The treatment output may be, for example, treatment output 124 in FIG. 1.

When the DRT approximation output 122 is the DRT attribution map 202, the treatment output 124 may be administered based on the DRT area approximation. For example, DRT area in the retina may indicate the presence of DME or AMD, and an appropriate treatment may be administered to treat DME or AMD. In some embodiments, DRT area in the retina that changes from a baseline timepoint over one or more timepoints after the baseline timepoint may indicate disease severity and/or treatment efficacy in subjects who are receiving a treatment. In some examples, appropriate treatment may be an anti-VEGF therapy, such as ranibizumab, aflibercept, or bevacizumab.

In some embodiments, the treatment output may be generated based on the approximate area of DRT as quantified by counting the number of high-importance pixels in the image input. In various embodiments, the treatment output may be generated based on changes in the approximate area of DRT in image inputs captured at timepoints after a baseline timepoint, relative to the baseline timepoint, of a retina of a patient. In such examples, the treatment output may further be generated based on changes in the area of DRT with respect to the approximated DRT locations captured at timepoints after the baseline timepoint, relative to the baseline timepoint, of the retina of the patient.

In some embodiments, the process 500 occurs after or in response to the patient of the process 400 and the patient associated with the image input, as a patient at high risk of experiencing or as a patient that is experiencing DRT at the step 408.

In some embodiments, the process 500 approximates DRT area in patients with better accuracy and consistency than expert human graders. The process 500 provides a technical effect of improving accuracy, reducing the overall computing resources, and/or reducing the time needed to provide an approximate area of DRT in subjects. Further, the process 500 may allow treatment outcomes in subjects to be generated more efficiently and accurately as compared to other methods and systems.

In some embodiments, the process 500 provides a technical improvement to the field of DRT measurement and/or the technical field of generating DRT treatment outputs. As noted above, the process 500 approximates DRT area in patients with better accuracy and consistency than expert human graders and may reduce the overall computing resources and/or time needed to approximate DRT area in subjects. In some embodiments, the process 500 includes a new combination of steps that results in the technical improvement over conventional DRT area approximation methods.

### V. Example Process for Approximation of DRT Volume

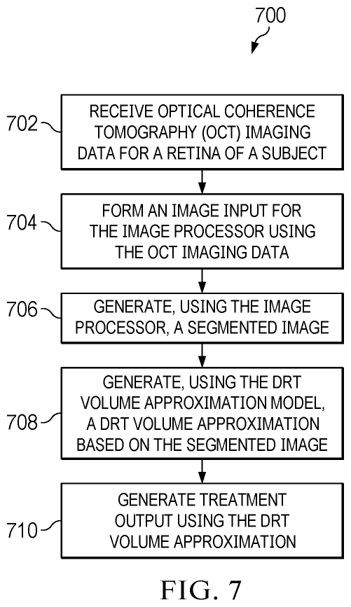

FIG. 7 is a flowchart of a process 700 for approximating the DRT volume using OCT images in accordance with various embodiments. A volume of DRT may be approximated in image inputs which have been classified as DRT-positive by generating a segmented OCT image and subtracting the volume of the retinal pathological elements (e.g., any cystic IRF and/or SRF) from the volume between two retinal layer elements. In various embodiments, process 500 is implemented using the DRT detection system 100, as described in FIG. 1, and more specifically, using DRT volume approximation model 120 as the DRT approximation model 112, as described in FIG. 3.

Step 702 of process 700 includes receiving optical coherence tomography (OCT) imaging data for a retina of a subject. The OCT imaging data may be, for example, OCT imaging data 132 in FIG. 1. The retina may be a retina diagnosed with or suspected of having a retinal disease. The retinal disease may be, for example, age-related macular degeneration (AMD), diabetic macular edema (DME), or some other type of retinal disease. In other embodiments, the retina may be a healthy retina or a retina for which no diagnosis has yet been made.

Step 704 of process 700 includes forming an image input for an image processor using the OCT imaging data. The image input may be, for example, processed image(s) 114 in FIG. 1. The image processor may be, for example, image processor 300 in FIG. 3. Step 704 may be performed in various ways. In one or more embodiments, forming the image input simply includes sending the OCT imaging data as is into the image processor 300. In other embodiments, forming the image input may include performing a set of preprocessing operations on the OCT imaging data using the image processor 108 of FIG. 1. The set of preprocessing operations may include, for example, without limitation, at least one of a normalization operation, a scaling operation, a resizing operation, a horizontal flipping operation, a vertical flipping operation, a cropping operation, a rotation operation, a noise filtering operation, or some other type of preprocessing operation.

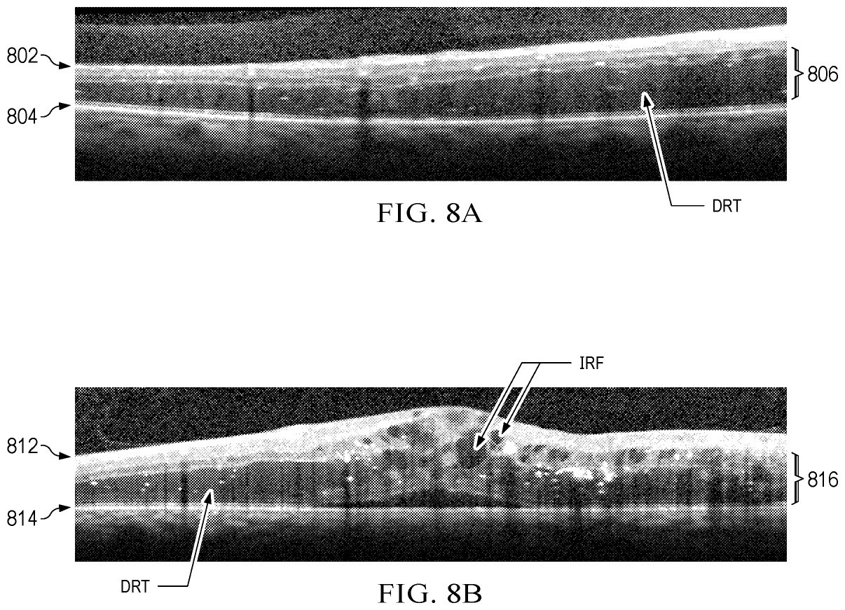

Step 706 of process 700 includes generating, via the image processor, a segmented image based on the image input. FIGS. 8A and 8B are annotated OCT B-scans which may be used, without the annotations, as image input (e.g., image input 102) and for which the trained DRT classification model (e.g., trained DRT classification model 110) has determined a DRT detection output (e.g., DRT detection output 116) as being DRT-positive. FIGS. 8A and 8B may be input for a DRT approximation model (e.g., DRT approximation model 112), specifically for a DRT volume approximation model 120, as discussed above in FIG. 3.

Step 708 of process 700 includes generating, via the DRT volume approximation model, a DRT volume approximation output using the segmented image(s). The DRT volume approximation model may be, for example, the DRT volume approximation model 120 in FIG. 3. The DRT volume approximation output may be, for example, the DRT volume approximation output 306 of FIG. 3. Once processed by image processor 300, various retinal elements are segmented from FIGS. 8A and 8B. In some embodiments, the retinal elements segmented may include retinal layer elements (such retinal layer elements 802 and 812 in FIGS. 8A and 8B, respectively, or retinal layer elements 804 and 814 in FIGS. 8A and 8B, respectively). In some embodiments, the retinal layers elements comprise an internal limiting membrane (ILM), Bruch's Membrane (BM), retinal pigment epithelium (RPE), ellipsoid zone (EZ), outer plexiform layer (OPL), external limiting membrane (ELM), retinal nerve fiber layer (RNFL), ganglion cell layer (GCL), inner plexiform layer (IPL), inner nuclear layer (INL), and/or outer plexiform layer (OPL). In various embodiments, the retinal elements segmented may include retinal pathological elements (such as intraretinal fluid (IRF), as annotated on FIG. 8B). In some embodiments, an approximation of volume, of the DRT (as indicated by 806 and 816 in FIGS. 8A and 8B, respectively) and healthy tissue combined (e.g., DRT volume approximation output 306), may be approximated by generating a volume between the two retinal layer elements surrounding the detected DRT. For example, in FIG. 8A, the volume of DRT and healthy tissue combined (e.g., 806) may be approximated by generating the volume between retinal layer elements 802 and 804. In some embodiments, the two retinal layer elements surrounding the detected DRT may include any combination of the retinal layers (e.g., ILM, BM, RPE, EZ, OPL, ELM, RNFL, GCL, IPL, INL, OPL). In some embodiments, the two retinal layer elements comprise the ILM and the BM. In other embodiments, the two retinal layer elements comprise the RPE and the EZ. In other embodiments, the two retinal layer elements comprise one of the BM, RPE, EZ, and ELM and one of the ILM, RNFL, GCL, IPL, INL, and OPL. In some embodiments, the DRT volume approximation model 120 is programed to approximate the volume of the DRT using the segmented image(s). In other embodiments, the volume of DRT and healthy tissue combined (e.g., DRT volume approximation output 306), may be generated by subtracting the volume of segmented retinal pathological elements from the volume between the two retinal layer elements surrounding the detected DRT. For example, in FIG. 8B, the volume of DRT and healthy tissue combined (e.g., 816) may be approximated by subtracting the volume of IRF from the volume between retinal layer elements 812 and 814.

Step 710 of the process 700 includes generating a treatment output using the DRT volume approximation. In one or more embodiments, the treatment output includes identifying the patient associated with the image input as a patient at high risk of experiencing DRT or as a patient that is experiencing DRT. In some embodiments, the treatment output may be, for example, the treatment output 124 of FIG. 1. When the DRT approximation output 122 is the DRT volume approximation output 306, the treatment output 124 may be administered based on the DRT volume approximation. For example, DRT volume in the retina may indicate the presence of DME or AMD, and an appropriate treatment may be administered to treat DME or AMD. In some embodiments, DRT volume in the retina that changes from a baseline timepoint over one or more timepoints after the baseline timepoint may indicate disease severity and/or treatment efficacy in subjects who are receiving a treatment. In some examples, appropriate treatment may be an anti-VEGF therapy, such as ranibizumab, aflibercept, or bevacizumab.

In some embodiments, the process 700 occurs after or in response to the patient of the process 400 and the patient associated with the image input, as a patient at high risk of experiencing or as a patient that is experiencing DRT at the step 408.

In some embodiments, the process 700 approximates DRT volume in patients with better accuracy and consistency than expert human graders. The process 700 provides a technical effect of improving accuracy, reducing the overall computing resources, and/or reducing the time needed to provide an approximate volume of DRT in subjects. Further, the process 700 may allow treatment outcomes in subjects to be generated more efficiently and accurately as compared to other methods and systems.

In some embodiments, the process 700 provides a technical improvement to the field of DRT measurement and/or the technical field of generating DRT treatment outputs. As noted above, the process 700 approximates DRT volume in patients with better accuracy and consistency than expert human graders and may reduce the overall computing resources and/or time needed to approximate DRT volume in subjects. In some embodiments, the process 700 includes a new combination of steps that results in the technical improvement over conventional DRT volume approximation methods.

### VI. Example Implementation of Computer System

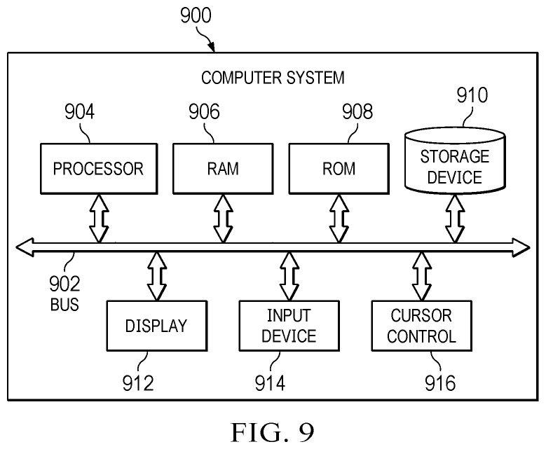

FIG. 9 is a block diagram of a computer system 900 in accordance with various embodiments. Computer system 900 may be an example of one implementation for computing platform 106 described in FIG. 1. In one or more examples, computer system 900 can include a bus 902 or other communication mechanism for communicating information, and a processor 904 coupled with bus 902 for processing information. In various embodiments, computer system 900 can also include a memory, which can be a random-access memory (RAM) 906 or other dynamic storage device, coupled to bus 902 for determining instructions to be executed by processor 904. Memory also can be used for storing temporary variables or other intermediate information during execution of instructions to be executed by processor 904. In various embodiments, computer system 900 can further include a read only memory (ROM) 908 or other static storage device coupled to bus 902 for storing static information and instructions for processor 904. A storage device 910, such as a magnetic disk or optical disk, can be provided and coupled to bus 902 for storing information and instructions.

In various embodiments, computer system 900 can be coupled via bus 902 to a display 912, such as a cathode ray tube (CRT) or liquid crystal display (LCD), for displaying information to a computer user. An input device 914, including alphanumeric and other keys, can be coupled to bus 902 for communicating information and command selections to processor 904. Another type of user input device is a cursor control 916, such as a mouse, a joystick, a trackball, a gesture input device, a gaze-based input device, or cursor direction keys for communicating direction information and command selections to processor 904 and for controlling cursor movement on display 912. This input device 914 typically has two degrees of freedom in two axes, a first axis (e.g., x) and a second axis (e.g., y), that allows the device to specify positions in a plane. However, it should be understood that input devices 914 allowing for three-dimensional (e.g., x, y, and z) cursor movement are also contemplated herein.

Consistent with certain implementations of the present teachings, results can be provided by computer system 900 in response to processor 904 executing one or more sequences of one or more instructions contained in RAM 906. Such instructions can be read into RAM 906 from another computer-readable medium or computer-readable storage medium, such as storage device 910. Execution of the sequences of instructions contained in RAM 906 can cause processor 904 to perform the processes described herein. Alternatively, hard-wired circuitry can be used in place of or in combination with software instructions to implement the present teachings. Thus, implementations of the present teachings are not limited to any specific combination of hardware circuitry and software.

In some embodiments, the network 104 may be implemented using a single network or multiple networks in combination. The network 104 may be implemented using any number of wired communications links, wireless communications links, optical communications links, or combination thereof. For example, in various embodiments, the network 104 may include the Internet or one or more intranets, landline networks, wireless networks, and/or other appropriate types of networks. In another example, the network 104 may comprise a wireless telecommunications network (e.g., cellular phone network) adapted to communicate with other communication networks, such as the Internet. In some cases, the network 104 includes at least one of a local area network (LAN), a virtual local area network (VLAN), a wide area network (WAN), a public land mobile network (PLMN), the Internet, or another type of network. The OCT imaging system 134 and the DRT detection system 100 may each include one or more electronic processors, electronic memories, and other appropriate electronic components for executing instructions such as program code and/or data stored on one or more computer readable mediums to implement the various applications, data, and steps described herein. For example, such instructions may be stored in one or more computer readable media such as memories or data storage devices (e.g., data storage 128) internal and/or external to various components of the DRT detection system 100, and/or accessible over the network 104.

The term “computer-readable medium” (e.g., data store, data storage, storage device, data storage device, etc.) or “computer-readable storage medium” as used herein refers to any media that participates in providing instructions to processor 904 for execution. Such a medium can take many forms, including but not limited to, non-volatile media, volatile media, and transmission media. Examples of non-volatile media can include, but are not limited to, optical, solid state, magnetic disks, such as storage device 910. Examples of volatile media can include, but are not limited to, dynamic memory, such as RAM 906. Examples of transmission media can include, but are not limited to, coaxial cables, copper wire, and fiber optics, including the wires that comprise bus 902.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 904 of computer system 900 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 900 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 900, whereby processor 904 would execute the analyses and determinations provided by these engines, subject to instructions provided by any one of, or a combination of, the memory components RAM 906, ROM 908, or storage device 910 and user input provided via input device 914.

### VII. Example Definitions and Content

The disclosure is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion.

In addition, as the terms “on,” “attached to,” “connected to,” “coupled to,” or similar words are used herein, one element (e.g., a component, a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

The term “subject” may refer to a subject of a clinical trial, a person undergoing treatment, a person undergoing anti-cancer therapies, a person being monitored for remission or recovery, a person undergoing a preventative health analysis (e.g., due to their medical history), or any other person or subject of interest. In various cases, “subject” and “subject” may be used interchangeably herein.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology, and toxicology are described herein are those well-known and commonly used in the art.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

As used herein, the term “about” used with respect to numerical values or parameters or characteristics that can be expressed as numerical values means within ten percent of the numerical values. For example, “about 50” means a value in the range from 45 to 55, inclusive.

The term “ones” means more than one.

As used herein, the term “plurality” can be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, a “model” may include one or more algorithms, one or more mathematical techniques, one or more machine learning algorithms, or a combination thereof.

As used herein, “machine learning” is the practice of using algorithms to parse data, learn from it, and then make a determination or prediction about something in the world. Machine learning uses algorithms that can learn from data without relying on rules-based programming.

As used herein, an “artificial neural network” or “neural network” (NN) refers to mathematical algorithms or computational models that mimic an interconnected group of artificial neurons that processes information based on a connectionistic approach to computation. Neural networks, which may also be referred to as neural nets, can employ one or more layers of linear units, nonlinear units, or both to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network processes information in two ways; when it is being trained it is in training mode and when it puts what it has learned into practice it is in inference (or prediction) mode. Neural networks learn through a feedback process (e.g., backpropagation) which allows the network to adjust the weight factors (modifying its behavior) of the individual nodes in the intermediate hidden layers so that the output matches the outputs of the training data. In other words, a neural network learns by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), a Squeeze and Excitation embedded neural network, a MobileNet, or another type of neural network.

As used herein, “deep learning” may refer to the use of multi-layered artificial neural networks to automatically learn representations from input data such as images, video, text, etc., without human provided knowledge, to deliver highly accurate predictions in tasks such as object detection/identification, speech recognition, language translation, etc.

### VIII. Recitation of Example Embodiments

Embodiment 1: A method comprising: receiving optical coherence tomography (OCT) imaging data for a retina of a subject; forming first image input for a machine learning model using the OCT imaging data; and generating, via the machine learning model, a diffuse retinal thickness (DRT) detection output based on the first image input, wherein the DRT detection output indicates whether or not a presence of DRT is detected in the retina of the subject.

Embodiment 2: The method of embodiment 1, wherein the DRT detection output is a positive detection when the machine learning model determines that a presence of diffuse retinal fluid indicates DRT and a negative detection when the machine learning model determines that DRT is not present.

Embodiment 3: The method of any one of embodiments 1-2, wherein the DRT detection output comprises at least one of: a probability value indicating the probability that DRT is present in the retina, a binary classification of DRT presence, or a value indicating an amount of diffuse retinal fluid in the retina.

Embodiment 4: The method of any one of embodiments 1-3, wherein preprocessing the OCT imaging data to generate the first image input comprises: performing a set of preprocessing operations on the OCT imaging data to form the first image input, the set of preprocessing operations comprising at least one of a normalization operation, a scaling operation, a resizing operation, a horizontal flipping operation, a vertical flipping operation, a cropping operation, a rotation operation, a noise filtering operation, or some other type of preprocessing operation.

Embodiment 5: The method of any one of embodiments 1-4, further comprising administering a treatment based on the DRT detection output.

Embodiment 6: The method of embodiment 5, wherein the treatment is an anti-VEGF therapy.

Embodiment 7: The method of any one of embodiments 1-6, further comprising: training the machine learning model using a training dataset that includes a plurality of training OCT images, wherein a training OCT image of the plurality of training OCT images is labeled as belonging to a category selected from a group consisting of positively present DRT, possibly present DRT, positively absent DRT, and ungradable.

Embodiment 8: The method of embodiment 7, wherein the plurality of training OCT images includes training OCT images labeled by a plurality of human graders and wherein the category selected for a particular training OCT image of the training OCT images by a majority of the plurality of human graders is used as ground truth for computing loss.

Embodiment 9: The method of any one of embodiments 1-8, further comprising: training the machine learning model using a training dataset that includes a plurality of training OCT images, wherein a training OCT image of the plurality of training OCT images is labeled as belonging to a category selected from a group consisting of DRT-positive, DRT-negative, and ungradable.

Embodiment 10: The method of any one of embodiments 1-9, further comprising: training the machine learning model using a training dataset that includes a plurality of training OCT images, wherein a training OCT image of the plurality of training OCT images is labeled as either DRT-positive or DRT-negative.

Embodiment 11: The method of any one of embodiments 1-10, wherein the machine learning model includes a deep learning model.

Embodiment 12: The method of any one of embodiments 1-11, wherein the machine learning model includes a convolutional neural network (CNN).

Embodiment 13: The method of any one of embodiments 1-12, wherein the first image input comprises an OCT B-scan; wherein the DRT detection output indicates the presence of DRT in the retina of the subject; and wherein the method further comprises: forming second image input for an image processor using the OCT B-scan; generating, using the image processor, a segmented OCT image; and generating, using a DRT volume approximation model, a DRT volume approximation based on the segmented image.

Embodiment 14: The method of embodiment 13, wherein the segmented OCT image identifies a first approximate volume of a retinal pathological element and identifies a second approximate volume between two retinal layer elements; wherein generating, using the DRT volume approximation model, the DRT volume approximation based on the segmented image comprises subtracting the first approximate volume from the second approximate volume; and wherein the difference between the first approximate volume and the second approximate volume is the DRT volume approximation.

Embodiment 15: The method of any one of embodiments 1-12, wherein the first image input comprises an OCT B-scan; wherein the DRT detection output indicates the presence of DRT in the retina of the subject; wherein the method further comprises generating, using a DRT mapping algorithm and the OCT B-scan, a DRT attribution map; and wherein the DRT attribution map indicates a region or location of the OCT B-scan that the machine learning model used in making the DRT detection output indicating the presence of DRT in the retina of the subject.

Embodiment 16: A method comprising: receiving optical coherence tomography (OCT) imaging data for a retina of a subject; forming an image input for a machine learning model using the OCT imaging data; generating, via the machine learning model, a diffuse retinal thickness (DRT) detection output based on the image input, wherein the DRT detection output indicates whether or not a presence of DRT is detected in the retina of the subject; and approximating an area of DRT present in the image input.

Embodiment 17: A method comprising: receiving optical coherence tomography (OCT) imaging data for a retina of a subject; forming an image input for a machine learning model using the OCT imaging data; generating, via the machine learning model, a diffuse retinal thickness (DRT) detection output based on the image input, wherein the DRT detection output indicates whether or not a presence of DRT is detected in the retina of the subject; and approximating a volume of DRT present in the image input.

Embodiment 18: The method of embodiment 16 or embodiment 17, further comprising administering a treatment based on the approximation of the area or the volume of DRT present in the image input.

Embodiment 19: The method of embodiment 18, wherein the treatment is an anti-VEGF therapy.

Embodiment 20: A system comprising: one or more data processors; and a non-transitory computer readable storage medium containing instructions which, when executed on the one or more data processors, cause the one or more data processors to perform part or all of one or more methods disclosed in embodiments 1-19.

Embodiment 21: A computer-program product tangibly embodied in a non-transitory machine-readable storage medium, including instructions configured to cause one or more data processors to perform part or all of one or more methods disclosed in embodiments 1-19.

### IX. Additional Considerations

While the present teachings are described in conjunction with various embodiments, it is not intended that the present teachings be limited to such embodiments. On the contrary, the present teachings encompass various alternatives, modifications, and equivalents, as will be appreciated by those of skill in the art.

For example, the flowcharts and block diagrams described above illustrate the architecture, functionality, and/or operation of possible implementations of various method and system embodiments. Each block in the flowcharts or block diagrams may represent a module, a segment, a function, a portion of an operation or step, or a combination thereof. In some alternative implementations of an embodiment, the function or functions noted in the blocks may occur out of the order noted in the figures. For example, in some cases, two blocks shown in succession may be executed substantially concurrently. In other cases, the blocks may be performed in the reverse order. Further, in some cases, one or more blocks may be added to replace or supplement one or more other blocks in a flowchart or block diagram.

Thus, in describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

1. A method comprising:
receiving optical coherence tomography (OCT) imaging data for a retina of a subject;
forming first image input for a machine learning model using the OCT imaging data; and
generating, via the machine learning model, a diffuse retinal thickness (DRT) detection output based on the first image input, wherein the DRT detection output indicates whether or not a presence of DRT is detected in the retina of the subject.

2. The method of claim 1, wherein the DRT detection output is a positive detection when the machine learning model determines that a presence of diffuse retinal fluid indicates DRT and a negative detection when the machine learning model determines that DRT is not present.

3. The method of claim 1, wherein the DRT detection output comprises at least one of: a probability value indicating the probability that DRT is present in the retina, a binary classification of DRT presence, or a value indicating an amount of diffuse retinal fluid in the retina.

4. The method of claim 1, wherein preprocessing the OCT imaging data to generate the first image input comprises:
performing a set of preprocessing operations on the OCT imaging data to form the first image input, the set of preprocessing operations comprising at least one of a normalization operation, a scaling operation, a resizing operation, a horizontal flipping operation, a vertical flipping operation, a cropping operation, a rotation operation, a noise filtering operation, or some other type of preprocessing operation.

5. The method of claim 1, further comprising administering a treatment based on the DRT detection output.

6. The method of claim 5, wherein the treatment is an anti-VEGF therapy.

7. The method of claim 1, further comprising:
training the machine learning model using a training dataset that includes a plurality of training OCT images, wherein a training OCT image of the plurality of training OCT images is labeled as belonging to a category selected from a group consisting of positively present DRT, possibly present DRT, positively absent DRT, and ungradable.

8. The method of claim 7, wherein the plurality of training OCT images includes training OCT images labeled by a plurality of human graders and wherein the category selected for a particular training OCT image of the training OCT images by a majority of the plurality of human graders is used as ground truth for computing loss.

9. The method of claim 1, further comprising:
training the machine learning model using a training dataset that includes a plurality of training OCT images, wherein a training OCT image of the plurality of training OCT images is labeled as belonging to a category selected from a group consisting of DRT-positive, DRT-negative, and ungradable.

10. The method of claim 1, further comprising:
training the machine learning model using a training dataset that includes a plurality of training OCT images, wherein a training OCT image of the plurality of training OCT images is labeled as either DRT-positive or DRT-negative.

11. The method of claim 1, wherein the machine learning model includes a deep learning model.

12. The method of claim 1, wherein the machine learning model includes a convolutional neural network (CNN).

13. The method of claim 1,
wherein the first image input comprises an OCT B-scan;
wherein the DRT detection output indicates the presence of DRT in the retina of the subject; and
wherein the method further comprises:
forming second image input for an image processor using the OCT B-scan;
generating, using the image processor, a segmented OCT image; and
generating, using a DRT volume approximation model, a DRT volume approximation based on the segmented image.

14. The method of claim 13,
wherein the segmented OCT image identifies a first approximate volume of a retinal pathological element and identifies a second approximate volume between two retinal layer elements;
wherein generating, using the DRT volume approximation model, the DRT volume approximation based on the segmented image comprises subtracting the first approximate volume from the second approximate volume; and
wherein the difference between the first approximate volume and the second approximate volume is the DRT volume approximation.

15. The method of claim 1,
wherein the first image input comprises an OCT B-scan;
wherein the DRT detection output indicates the presence of DRT in the retina of the subject;
wherein the method further comprises generating, using a DRT mapping algorithm and the OCT B-scan, a DRT attribution map; and
wherein the DRT attribution map indicates a region or location of the OCT B-scan that the machine learning model used in making the DRT detection output indicating the presence of DRT in the retina of the subject.

16. A method comprising:
receiving optical coherence tomography (OCT) imaging data for a retina of a subject;
forming an image input for a machine learning model using the OCT imaging data;
generating, via the machine learning model, a diffuse retinal thickness (DRT) detection output based on the image input, wherein the DRT detection output indicates whether or not a presence of DRT is detected in the retina of the subject; and
approximating an area of DRT present in the image input.

17. A method comprising:
receiving optical coherence tomography (OCT) imaging data for a retina of a subject;
forming an image input for a machine learning model using the OCT imaging data;
generating, via the machine learning model, a diffuse retinal thickness (DRT) detection output based on the image input, wherein the DRT detection output indicates whether or not a presence of DRT is detected in the retina of the subject; and
approximating a volume of DRT present in the image input.

18. The method of claim 16, further comprising administering a treatment based on the approximation of the area present in the image input.

19. The method of claim 18, wherein the treatment is an anti-VEGF therapy.

20. A system comprising: one or more data processors; and a non-transitory computer readable storage medium containing instructions which, when executed on the one or more data processors, cause the one or more data processors to perform part or all of the method disclosed in claim 1.

21. A computer-program product tangibly embodied in a non-transitory machine-readable storage medium, including instructions configured to cause one or more data processors to perform part or all of the method disclosed in claim 1.

22. The method of claim 17, further comprising administering a treatment based on the volume of DRT present in the image input.

23. The method of claim 22, wherein the treatment is an anti-VEGF therapy.
